(19) United States
(12) Patent Application Publication
(10) Pub. No.: US 2009/0036889 A1
(43) Pub. Date: Feb. 5, 2009

(54) METHOD AND APPARATUS FOR TREATMENT OF SLEEP APNEA

(76) Inventor: R. Sam Callender, Golden, CO (US)

Correspondence Address: DORR, CARSON & BIRNEY, P.C.;ONE CHERRY CENTER, 501 SOUTH CHERRY STREET, SUITE 800, DENVER, CO, 80246, US

(21) Appl. No.: 11/830,335

(22) Filed: Jul. 30, 2007

(51) Int. Cl. A61B 17/00

(52) U.S. Cl. 606/55; 128/898; 623/17.17

(57) ABSTRACT

A method and apparatus for treatment of sleep apnea employs bone screws implanted into a patient's anterior maxillary bone above and posterior to the cuspids, and posterior mandibular bone below and between any of the posterior teeth. Elastics are stretched between the maxillary and mandibular bone screws to exert forces to bias the mandible forward with respect to the maxilla. An aligner can be placed between the patient's upper and lower to help maintain proper positioning of the mandible.

### BACKGROUND OF THE INVENTION

1. Field of the Invention

The present invention relates generally to the field of methods and devices for treatment of sleep apnea and related sleep disorders. More specifically, the present invention discloses a method and apparatus for treatment of sleep apnea and related sleep disorders using bone screws attached to the mandible and maxilla, with elastics extending between the bone screws to move the mandible forward relative to the maxilla.

2. Statement of the Problem

Sleep disorders, such as sleep apnea, snoring and bruxism, can have potentially serious health and social consequences, including daytime fatigue, a compromised immune system, poor mental and emotional health, irritability and lack of productivity. These sleep disorders have also been linked to an increased risk of diabetes, high blood pressure, stroke and heart attacks. Snoring and sleep apnea are both generally caused by blockage of the pharyngeal airway by excess tissue when the muscles associated with the tongue, mandible and soft palate relax during sleep. As the tongue relaxes, it tends to move posteriorly and can block the airway. Snoring is often caused by partial obstruction of breathing during sleep. In contrast, sleep apnea occurs when the tongue and soft palate collapse posteriorly and completely block the airway.

Many approaches have been tried in the past to treat sleep apnea and snoring. Various types of surgery, such a uvulapharyngoplasty and other types of surgery of the soft palate, oropharynx and nasopharynx have using in treating these conditions. However, any type of invasive surgery has obvious risks and disadvantages.

The prior art in this field also includes a variety of intraoral dental appliances and mandibular advancement devices, such as disclosed in U.S. Patent Application Publication No. 2007/0079833 (Lamberg), U.S. Pat. Nos. 5,365,945 and 6,729,335 (Halstrom) and others. These devices typically employ one or more polymeric dental appliances (e.g., bite trays, retainers, or splints) that fit over or contact a patient's teeth to shift the mandible forward relative to the maxilla to keep the airway open during sleep. However, since the forces used to reposition the mandible are carried by the teeth, these forces can also cause undesired repositioning of the teeth as well. In addition, many conventional dental appliances are relatively bulky and obtrusive, which interferes with the patient's ability to sleep and can result in poor patient compliance.

U.S. Pat. No. 6,109,265 (Frantz et al.) discloses a dental appliance with upper and lower plastic trays that conform to the patient's upper and lower teeth, soft tissue and palate. Elastic bands extend between pairs of retention hooks on the upper and lower trays to pull the mandible forward. Here again, the forces used to reposition the mandible are largely carried by the teeth.

U.S. Pat. No. 6,983,752 (Garabadian) discloses another example of a dental appliance with upper and lower trays for treatment of sleep disorders. Bite pads attached to the upper and lower trays allow limited vertical and lateral movement, while maintaining the occlusal surfaces of the trays in a predetermined spaced relationship. A number of buttons are attached to the buccal surfaces of the trays to attach elastic bands extended between the upper and lower trays.

Herbst appliances are commonly used in orthodontics to reposition the mandible in a more forward position to treat over-bite conditions. An example of a Herbst appliance is disclosed in U.S. Patent Application Publication No. 2006/0234180 (Huge et al.). A Herbst mechanism typically spans between the upper posterior teeth and the lower canine region. One common configuration uses a two-part telescoping mechanism consisting of a rod connected to the patient's lower arch and a tube connected to the upper arch. The ends of these telescoping segments have eyelets engaging pivots secured to orthodontic bands on the patient upper and lower arches. As the patient closes his or her teeth, the telescoping mechanism slides together until a predetermined limit is reached. Beyond that limit, the telescoping segments exert a force that tends to reposition the mandible forward with respect to the maxilla. Here again, the forces for repositioning the mandible are carried by the patient's teeth, and can undesirably change the positions of the patient's teeth as well.

3. Solution to the Problem

The present invention addresses the shortcomings associated the prior art in this field by employing bone screws connected by elastics to transmit the forces used to reposition the mandible directly to the bone structures of the mandible and maxilla, with only incidental forces being carried by the teeth. In addition, an aligner can be placed between the patient's upper and lower teeth to help ensure proper positioning of the mandible. This aligner can be made much smaller, lighter and less obtrusive due to the minor forces involved.

### SUMMARY OF THE INVENTION

This invention provides a method and apparatus for treatment of sleep apnea and snoring employing bone screws implanted into a patient's anterior maxillary bone and posterior mandibular bone. For example, the maxillary bone screws can be implanted below the crown and between the teeth at the mucosal-gingival junction above and posterior to the cuspids, and the mandibular bone screws can be implanted below any of the posterior teeth. Elastics are stretched between the maxillary and mandibular bone screws to exert forces to bias the mandible forward with respect to the maxilla. An aligner can be placed between the patient's upper and lower to help maintain proper positioning of the mandible in the forward position.

These and other advantages, features, and objects of the present invention will be more readily understood in view of the following detailed description and the drawings.

### BRIEF DESCRIPTION OF THE DRAWINGS

The present invention can be more readily understood in conjunction with the accompanying drawings, in which:

FIG. 1 is a side perspective view of a patient's dental anatomy showing upper and lower bone screws 20 and 22 connected by an elastic 25, and also showing an aligner 30.

FIG. 2 is a perspective view of a bone screw.

FIG. 3 is an exploded perspective view of the upper and lower sections 31, 32 of an aligner 30.

FIG. 4 is a vertical cross-sectional view of an aligner 30 with upper and lower molars.

FIG. 5 is a top view of the lower section 32 of an aligner 30 on a patient's lower teeth.

FIG. 6 is a side view of an embodiment of an aligner 30 with a screw adjustment mechanism allowing a range of anterior-posterior movement between the upper and lower sections 31, 32 of the aligner 30.

FIG. 7 is a side view of another embodiment of an aligner 30 with another type of screw adjustment mechanism.

FIG. 8 is a side view of a patient's dental anatomy with multiple lower bone screws.

FIG. 9 is a side view of a patient's dental anatomy showing another arrangement for attaching an elastic 25 between a maxillary bone screw 20 and multiple mandibular bone screws 22a-22e.

FIG. 10 is a side view of a patient's dental anatomy showing another arrangement for attaching an elastic 25 between multiple maxillary bone screws 20a-20e and mandibular bone screws 22a-22e.

### DETAILED DESCRIPTION OF THE INVENTION

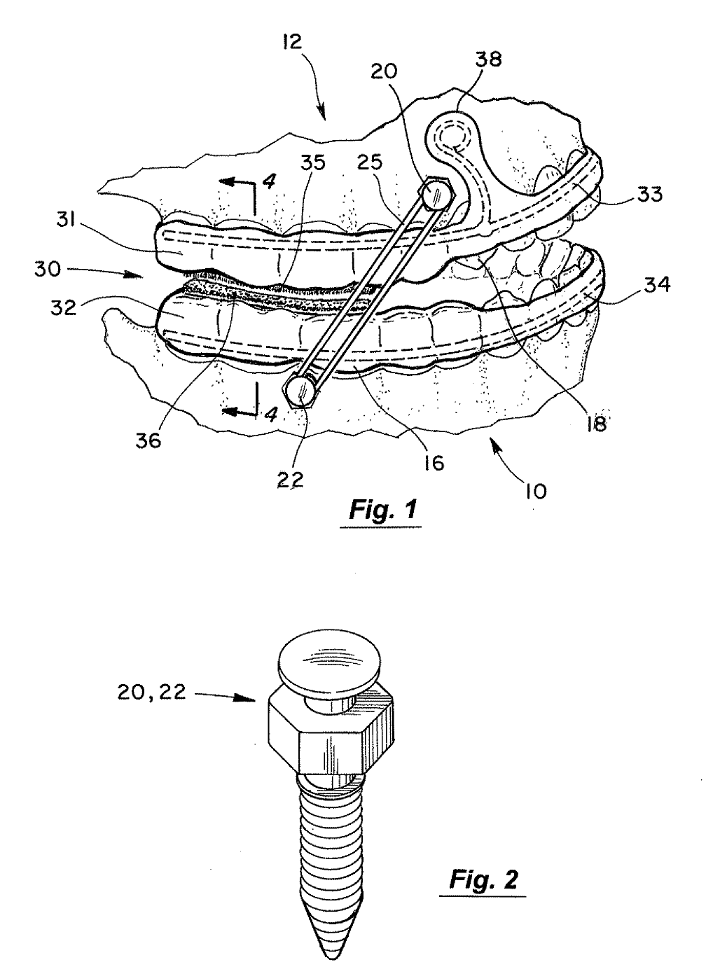

Turning to FIG. 1, a side perspective view of a patient's dental anatomy is provided showing the major components of the present invention. A maxillary bone screw 20 is attached to the basal bone of the anterior portion of the maxilla 12. For example, maxillary bone screws 20 can be attached above and posterior to the upper cuspids 18 or bicuspids below the crown and between the teeth at the mucosal-gingival junction as shown in FIG. 1. The maxillary bone screws 20 can also be implanted behind and above the patient's molars. Similarly, mandibular screws 22 are implanted in the basal bone structure of the posterior portion of the mandible 10′ typically below and between the first and second molars 16. However, the mandibular screws 22 could be implanted below any of the patient's lower molars, bicuspids, cuspids, or other posterior teeth. One pair of maxillary and mandibular bone screws 20, 22 would usually be attached on the left side of the patient and a second pair of bone screws would be attached on the right side for left-right symmetry.

A number of bone screws are commercially available and have long been used, for example, to anchor orthodontic archwires. FIG. 2 is a perspective view of one type of bone screw. Most conventional bone screws include a screw portion that can be threaded into the bone, and a head portion extending beyond the soft tissue covering the bone that can be rotated with a dental tool. Optionally, the head of each bone screw 20, 22 can include an annular recess to hold an elastic 25. The threaded portions of the bone screws can be implant into the basal bone structures of the maxilla 12 and mandible 10 using conventional techniques. A small initial incision is often made through the overlaying soft tissue. Holes for the bone screws can be drilled and tapped in the bone, although many bone screws are self-drilling and self-tapping.

After installation, the head of each bone screw typically extends outward in the buccal direction beyond the surface of the soft tissue covering the bone so that elastics to be easily attached to the exposed heads of the bone screws. Alternatively, the bone screws could be attached on the lingual side of the dental arches with the heads of the bone screws extending in the lingual direction, although this arrangement may have the disadvantage of crowding the tongue 14. After installation of the bone screws, elastics 25 are stretched between the pairs of maxillary and mandibular bone screws 20, 22 to exert forces that tend move the mandible 10 forward and upward with respect to the maxilla 12. It should be noted that the major forces of repositioning the jaw are carried by the bone structures of the maxilla 12 and mandible 10, rather than the teeth.

For example, conventional orthodontic elastic bands 25 (e.g., class 2 bands) can be used for this purpose. It should be noted that a progressive series of bands of different mechanical properties can be used over time. These elastics 25 can be easily attached to the bone screws 20, 22 by the patient before going to sleep and then removed after waking. Multiple elastics can also be attached between pairs of maxillary and mandibular bone screws 20, 25, if desired. Other types of elastic members could be substituted.

Optionally, an aligner 30 can be placed between the patient's upper and lower teeth to ensure proper positioning of the mandible 10 with respect to the maxilla 12. The aligner 30 can be made of a polymeric material (e.g., acrylic) using conventional orthodontic techniques. The upper and lower surfaces of the aligner 30 incorporate a series of recesses to receive the patient's upper and lower teeth. Since only nominal forces are carried by the aligner 30 and teeth, the aligner 30 can have a very light construction with a minimal thickness sufficient to contact the cusps of the teeth.

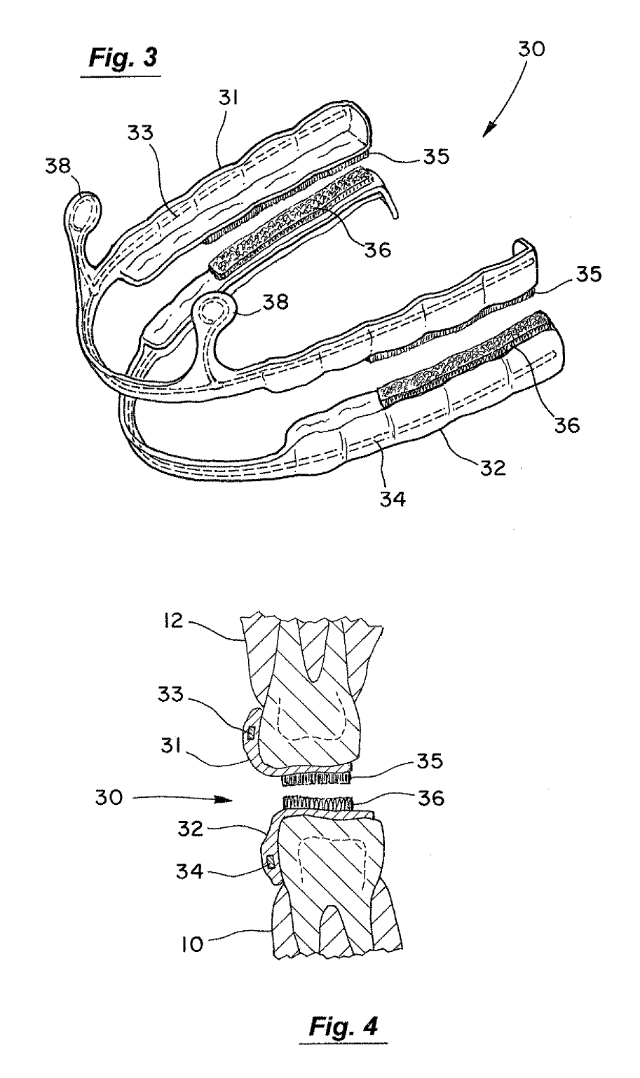

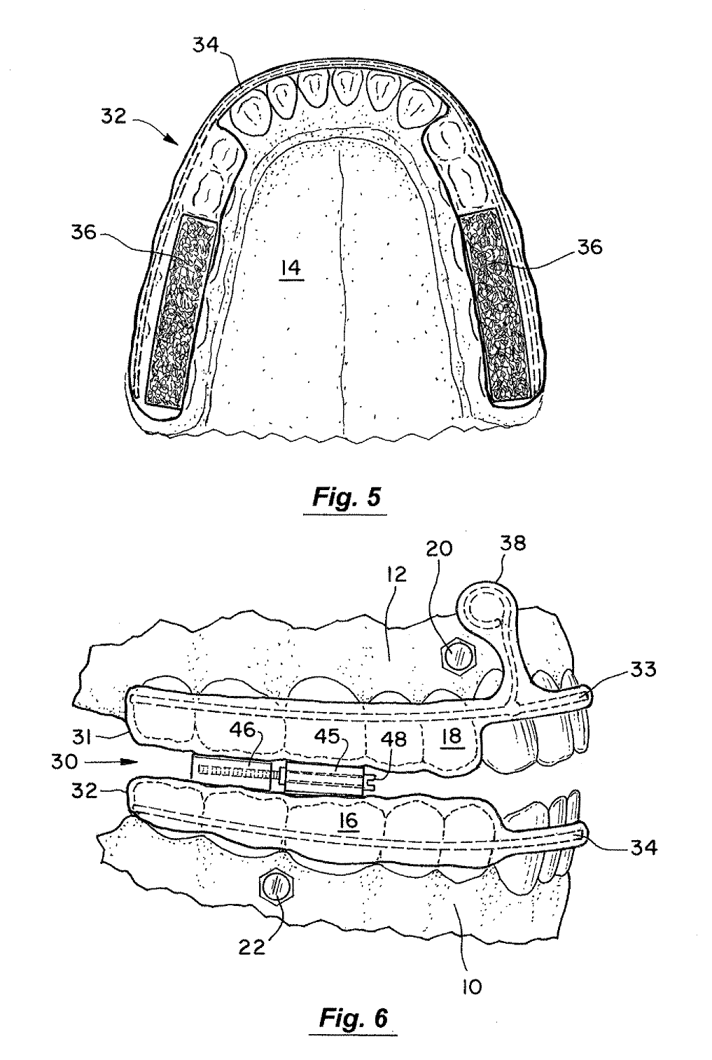

The embodiment of the aligner 30 shown in the exploded perspective view illustrated in FIG. 3 has a two-piece construction that includes an upper section 31 and a lower section 32, with an adjustment mechanism to provide a degree of adjustability along an anterior-posterior axis between the upper and lower sections 31, 32. This in turn provides a degree of adjustability between the dental arches. The upper section 31 of the aligner has a series of recesses intended to receive the patient's upper teeth, while the lower section 32 has a series of recesses to receive the patient's lower teeth. FIG. 5 is a top view of the lower section 32 of an aligner 30 on a patient's lower teeth. It should be understood that the upper and lower sections 31, 32 are separate in this embodiment, but work together to function as a single aligner to maintain a desired positioning between the patient's maxilla and mandible.

In this embodiment, the occlusal surfaces of the upper and lower sections 31, 32 of the aligner 30 bear complementary patches 35, 36 of a hook-and-loop fastener material (e.g., Velcro® material) that removably secure the upper and lower sections 31, 32 together FIG. 4 is a corresponding vertical cross-sectional view of the aligner 30 including upper and lower molars. Over the course of treatment, the healthcare professional can separate the fastener patches 35, 36 and adjust the anterior-posterior positioning the upper and lower sections 31, 32 of the aligner 30 to accommodate changes in the patient's jaw position. Optionally, a numbers of lines or other visual indicia can be placed on the upper and lower sections 31, 32 of the aligner 30 to assist the healthcare provider in measuring how much adjustment has been made over the course of treatment.

It should be understood that other types of adjustment mechanisms could be substituted to adjust the anterior-posterior positions of the upper and lower sections 31, 32 of the aligner 30. For example, FIG. 6 is a side view of an embodiment of an aligner 30 with a screw adjustment mechanism allowing a range of anterior-posterior movement. A first block 45 is secured to the occlusal surface of the upper section 31 of the aligner, and a second block 46 is secured to the occlusal surface of the lower section 32 of the aligner 30. An adjustment screw 48 is inserted through holes aligned in both blocks 45, 46. The threaded portion of the screw 48 engages corresponding threads in the second block 46, while the head of the screw 48 remains accessible on the anterior face of the first block 45. This enables the healthcare provider to adjust the spacing between the blocks 45, 46 by using a small screw driver to turn the head of the adjustment screw 48. The blocks 45 and 46 also serve as bite blocks to maintain a desired vertical separation between the patient's upper and lower teeth while the aligner 30 is in place. Optionally, a number of reinforcing pins can be placed in sliding engagement with the blocks 45, 46 parallel to the axis of the adjustment screw 48 for additional support.

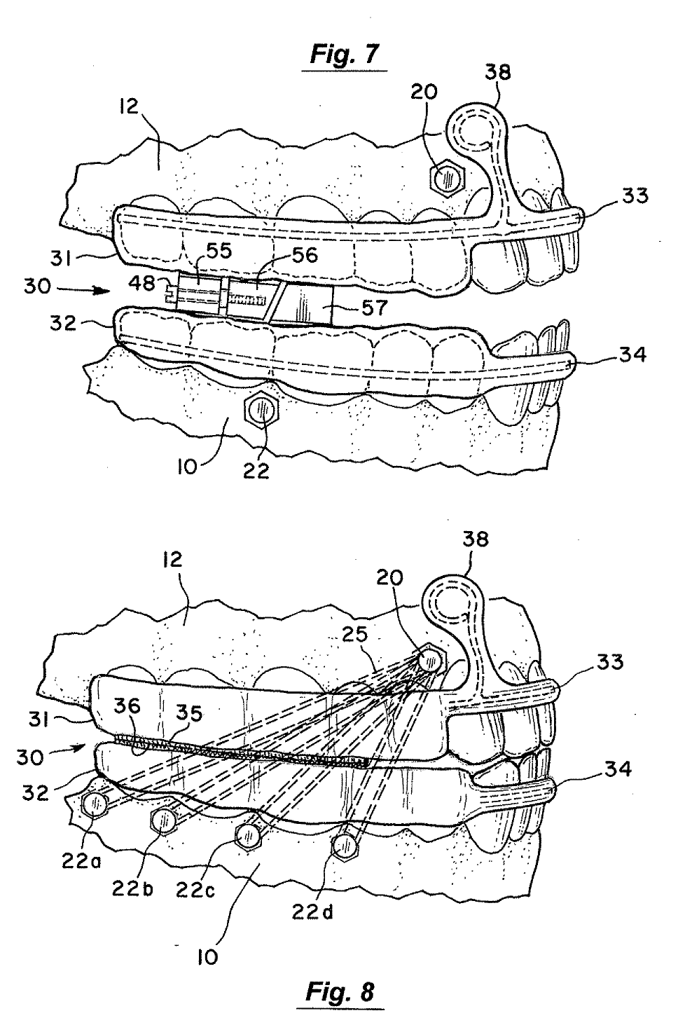

FIG. 7 is a side view of another embodiment of an aligner 30 with a screw adjustment mechanism employing bite blocks 55, 56 and 57 with angled surfaces that allow the patient to open and close the jaw. Here again, an adjustment screw 48 enables the healthcare provider to adjust the anterior-posterior spacing between blocks 55 and 56. The complementary angled surfaces on blocks 56 and 57 guide the upper and lower sections 31, 32 of the aligner 30 into the desired anterior-posterior relationship when the jaw is closed.

As previously noted, a primary advantage of the present invention is that the forces used to reposition the jaw are carried by the bone screws attached to the maxilla and mandible, rather than be carried by the teeth. However, it should be understood that the present invention provides an additional advantage in that the aligner 30 can be made lighter and thinner due to the minimal forces that it carries. For example, the aligner 30 can be configured to primarily engage the patient's posterior teeth (i.e., molars and bicuspids). Optionally, the anterior portion of the aligner 30 can be reduced in size or replaced with a labial bow. In addition, the aligner 30 can be designed to extend primarily on the buccal and occlusal aspects of the teeth, as shown in the vertical cross-sectional view depicted in FIG. 4. This essentially eliminates intrusion of the aligner on the lingual aspect of the dental arch, reduces infringement on tongue space, and thereby enhances patient comfort.

The embodiment of the aligner 30 shown in FIGS. 1, 3 and 5 includes upper and lower labial bows 33, 34 for increased strength and rigidity. Mesh or other reinforcing members can be embedded in the aligner for added strength. In the embodiment shown in the drawings, the anterior portion of the aligner 30 includes a thin layer of acrylic material covering the upper and lower labial bows 33, 34 adjacent to the labial surfaces of the anterior teeth to prevent irritation of the patient's lips by the labial bows 33, 34.

Optionally, an extension or shield 38 can extend upward on the labial aspect of the maxillary anterior portion of the aligner 30 to above the maxillary bone screw 20 to protect the soft tissue of the lip from irritation by the bone screw 20 and elastic 25. For example, the extension 28 can be a paddle-shaped member made of acrylic with an internal wire reinforcement soldered or welded to the upper labial bow 33. The extension 38 should preferably have a sufficient thickness to lift the soft tissue of the lip away from excessive contact with the head of the bone screw 20 and elastic 25.

Returning to the embodiment of the aligner 30 shown in FIGS. 1 and 3, it should be noted that the forces exerted by the elastics 25 may tend to pull the lower section 32 of the aligner forward and out of contact with the lower teeth. The lower section 32 can include a posterior flange or surface as shown in FIG. 3 that extends around the distal aspect of the last tooth to provide additional retention. The aligner 30 can also be equipped with a number of ball clasps, or fingers that extend into the interproximal spaces between the patient's teeth to removably secure the aligner 30 in place.

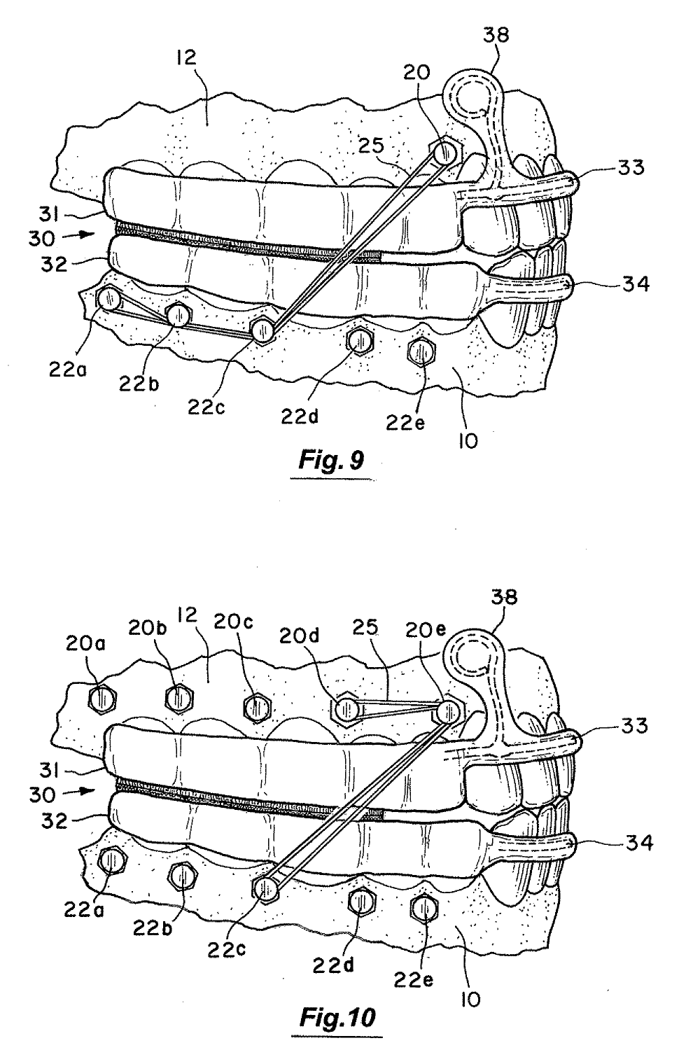

FIG. 8 is a side view of a patient's dental anatomy showing an implementation of the present invention using multiple mandibular bone screws 22a-22d. This configuration can be used to allow multiple elastics 25 to be stretched between the maxilla 12 and mandible 10, and/or to change the angle of the force exerted by an elastic. FIG. 9 is a side view of a patient's dental anatomy showing an alternative arrangement for attaching an elastic 25 using multiple mandibular bone screws 22a-22e. FIG. 10 is a side view of showing another arrangement for attaching an elastic 25 using multiple maxillary bone screws 20a-20e and mandibular bone screws 22a-22e. This configuration allows the elastics 25 to exert greater force biasing the patient's jaw toward a closed position and also allows a greater range of motion.

The above disclosure sets forth a number of embodiments of the present invention described in detail with respect to the accompanying drawings. Those skilled in this art will appreciate that various changes, modifications, other structural arrangements, and other embodiments could be practiced under the teachings of the present invention without departing from the scope of this invention as set forth in the following claims.

I claim:

1. A method for treatment of sleep apnea comprising:
implanting a maxillary bone screw into a patient's anterior maxillary bone;
implanting a mandibular bone screw into a patients posterior mandibular bone; and
removably attaching an elastic member between the maxillary bone screw and the mandibular bone screw for exerting forces to bias the mandible forward with respect to the maxilla.

2. The method of claim 1 further comprising placing an aligner between the patient's upper and lower teeth to maintain a desired positioning between the patient's maxilla and mandible.

3. The method of claim 1 wherein the bone screws extend outward in the buccal direction.

4. The method of claim 1 wherein the elastic member comprises an elastic band.

5. The method of claim 1 wherein the maxillary bone screw is implanted above and behind the patient's upper cuspid.

6. The method of claim 1 wherein the maxillary bone screw is implanted above and behind the patients upper bicuspid.

7. The method of claim 1 wherein the mandibular bone screw is implanted below the patient's lower molars.

8. An apparatus for treatment of sleep apnea comprising:
a maxillary bone screw implanted into a patient's anterior maxillary bone;
a mandibular bone screw implanted into a patient's posterior mandibular bone;
an elastic member removably attached between the maxillary bone screw and the mandibular bone screw for exerting forces to bias the mandible forward with respect to the maxilla; and
an aligner placed between the patient's upper and lower teeth to maintain a desired positioning between the patient's maxilla and mandible.

9. The apparatus of claim 8 wherein the bone screws extend outward in the buccal direction.

10. The apparatus of claim 8 wherein the elastic member comprises an elastic band.

11. The apparatus of claim 8 wherein the maxillary bone screw is implanted above and behind the patient's upper cuspid.

12. The apparatus of claim 8 wherein the maxillary bone screw is implanted above and behind the patient's upper bicuspid.

13. The apparatus of claim 8 wherein the mandibular bone screw is implanted below the patient's lower molars.

14. The apparatus of claim 8 wherein the mandibular bone screw is implanted below the patient's lower bicuspid.

15. The apparatus of claim 8 wherein the aligner further comprises:
an upper section to receive the patient's upper teeth;
a lower section to receive the patient's lower teeth; and
an adjustment mechanism removably securing the upper section to the lower section and providing a degree of adjustability along an anterior-posterior axis between the upper section and lower section.

16. The apparatus of claim 15 wherein the adjustment mechanism comprises complementary patches of hook-and-loop fastener material on the occlusal surfaces of the upper section and lower section.

17. The apparatus of claim 15 wherein the adjustment mechanism comprises:
a first block secured to the occlusal surface of the upper section;
a second block secured to the occlusal surface of the lower section; and
an adjustment screw extending between the first block and second block to adjust the spacing between the first block and the second block.

18. The apparatus of claim 15 wherein the aligner further comprises an extension extending from the upper section of the aligner to above the maxillary bone screw to protect the soft tissue of the lip from irritation by the maxillary bone screw and elastic member.
